(19) United States
(12) Patent Application Publication
(10) Pub. No.: US 2008/0124406 A1
(43) Pub. Date: May 29, 2008

(54) Spray Mixture For Soothing A Cow Udder

(76) Inventor: Mart M. Prost, Williamsburg (CA)

Correspondence Address: DONALD J. ERSLER, S.C., 725 GARVENS AVENUE, BROOKFIELD, WI, 53005, US

(21) Appl. No.: 11/849,071

(22) Filed: Aug. 31, 2007

Related U.S. Application Data
(63) Continuation-in-part of application No. 11/604,465, filed on Nov. 27, 2006, now abandoned.

(51) Int. Cl. A61K 36/82; A61P 43/00

(52) U.S. Cl. 424/729

(57) ABSTRACT

An udder soothing mixture preferably includes water, peppermint oil, menthol, tea tree oil, an emulsifying agent and a carrier. The peppermint oil, menthol and tea tree oil are organic active ingredients. The emulsifying agent is preferably Simulgel, but other emulsifying agents may also be used. The carrier is preferably Glycerin USP, but other carriers may also be used. The peppermint oil, menthol, tea tree oil and Glycerin USP are mixed together. The Glycerin USP absorbs the peppermint oil, menthol and tea tree oil. The Glycerin USP mixture is combined with Simulgel. Water is then added to the Simulgel mixture. The udder soothing mixture may be sprayed by a manual or automated sprayer. It is preferable to add coloring to udder soothing mixture.

### CROSS-REFERENCES TO RELATED APPLICATIONS

This is a continuation-in-part patent application taking priority from nonprovisional application Ser. No. 11/604,465 filed on Nov. 27, 2006.

### BACKGROUND OF THE INVENTION

1. Field of the Invention

The present invention relates generally to cow udders and more specially to a spray mixture for soothing a cow udder that may be applied without making physical contact with the cow.

2. Discussion of the Prior Art

There are numerous lotions, which are used to sooth the udder of a cow, after long hours of milk production. The numerous lotions must be applied by hand and cannot be sprayed, due to the viscosity thereof. Applying the lotion by hand is time consuming for a large dairy operation. Udder Comfort of Ontario, Canada does offer a soothing lotion that includes peppermint oil, tea tree oil and menthol. The emulsification system in the Udder Comfort lotion is a combination of emulsifying agents, namely Peg 100 Stearate, Steareth 21, Steareth 2, TriethanolamineStearic acid soap and acrylates/C10-30 alkyl acrylate crosspolymer. However, the Udder Comfort soothing lotion cannot be sprayed, even after reducing the viscosity thereof. Therefore, it appears the prior art does not disclose a mixture for soothing a cow udder that may be sprayed on the udder.

Accordingly, there is a clearly felt need in the art for a spray mixture for soothing a cow udder, which may be applied without having to physically contact the cow.

### SUMMARY OF THE INVENTION

The present invention is particularly directed to providing a spray mixture, which may be applied without making physical contact with the cow. The spray mixture for soothing a cow udder (udder soothing mixture) preferably includes water, peppermint oil, menthol, tea tree oil, an emulsifying agent and a carrier. The peppermint oil, menthol and tea tree oil are organic active ingredients. The emulsifying agent is preferably Simulgel, but other emulsifying agents may also be used. It is preferable to use Simulgel NS. Simulgel is a tradename. The carrier is preferably Glycerin USP, but other carriers may also be used, such as humectants, glycols, alcohols, polyols and silicones. The peppermint oil, menthol, tea tree oil and Glycerin USP are mixed together. The Glycerin USP absorbs the peppermint oil, menthol and tea tree oil. The Glycerin USP mixture is combined with Simulgel to form Simulgel mixture. Water is then added to the Simulgel mixture. The udder soothing mixture may be sprayed by a mechanical pump spray commonly found in household cleaning bottles or any other suitable spray system. It is preferable to add color to the udder soothing mixture with a food dye or an organic dye. The color would act as evidence that an udder had been sprayed with the udder soothing mixture.

Although specific components have been disclosed in the udder soothing mixture, the udder soothing mixture should not be limited to those components, but should include any component that may substituted for any of the previously recited component.

Accordingly, it is an object of the present invention to provide an udder soothing mixture, which may be applied without having to physically contact the cow.

These and additional objects, advantages, features and benefits of the present invention will become apparent from the following specification.

### DETAILED DESCRIPTION OF THE PREFERRED EMBODIMENTS

The following udder soothing mixture describes preferred components for soothing a cow udder. The udder soothing mixture preferably includes water, peppermint oil, menthol, tea tree oil, an emulsifying agent and a carrier. The peppermint oil, menthol and tea tree oil are organic active ingredients. The emulsifying agent is preferably Simulgel, but other emulsifying agents may also be used. The carrier is preferably Glycerin USP, but other carriers may also be used, such as humectants, glycols, alcohols, polyols and silicones.

Other glycerins may also be used, such as ethylhexyl glycerin. The glycols that may be used include propylene glycol, butylene glycol, hexylene glycol, pentylene glycol, isoprene glycol, caprylyl glycol, dipropylene glycol and ethoxydiglycol. The alcohols that may be used include ethanol and Isopropyl alcohol. The polyols that may be used include sorbitol and xylitol. The silicones that may be used include cyclomethicone and dimethicone copolyol.

It is preferable to add color to the udder soothing mixture with a food dye or an organic dye. The color would act as evidence that an udder had been sprayed with the udder soothing mixture. The food or organic dye is blended in a quantity of water to form tinted water. The tinted water is then added to the regular quantity of water.

Preferred percentages of each of the components are disclosed in the below table:

Udder Soothing Mixture

| Component | Percent by weight |
|---|---|
| Water | 10.0-98.7 |
| Glycerin USP | 0.5-30.0 |
| Peppermint Oil | 0.1-25.0 |
| Menthol | 0.1-25.0 |
| Tea Tree Oil | 0.1-25.0 |
| Simulgel | 0.5-5.0 |
| Coloring | 0.00001-5.0 |

The peppermint oil, menthol, tea tree oil and Glycerin USP are mixed together. The Glycerin USP absorbs the peppermint oil, menthol and tea tree oil. The Glycerin USP, peppermint oil, menthol, tea tree oil mixture is combined with Simulgel. Water is then added to the Simulgel mixture. The Simulgel suspends the Glycerin USP, peppermint oil, menthol, tea tree oil mixture in the water.

The udder soothing mixture may be sprayed by a mechanical pump spray, similar to those found in U.S. Pat. Nos. 4,273,268 and 4,944,431. Applicant hereby incorporates by reference the disclosure found in U.S. Pat. Nos. 4,273,268 and 4,944,431. However, other types of spraying devices, such as aerosol sprayers or automated sprayers may also be used to spray the udder soothing mixture on a cow udder.

While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

I claim:

1. A method for soothing a cow udder, comprising the steps of:
mixing peppermint oil, menthol and tea tree oil in a carrier to form a carrier mixture;
mixing said carrier mixture in an emulsifying agent to form an emulsifying mixture;
adding water to said emulsifying mixture to form a udder soothing mixture; and
spraying said udder smoothing mixture on at least one cow udder.

2. The method for soothing a cow udder of claim 1, further comprising the steps of:
providing said carrier taken from the group of glycerin, humectants, glycols, alcohols, polyols and silicones, said carrier being 0.5-30.0 parts by weight of a total of said udder soothing mixture.

3. The method for soothing a cow udder of claim 1, further comprising the steps of:
supplying said emulsifying agent in an amount of 0.5-5.0 parts by weight of a total of said udder soothing mixture.

4. The method for soothing a cow udder of claim 1, further comprising the steps of:
supplying said peppermint oil in an amount of 0.1-25.0 parts by weight of a total of said udder soothing mixture.

5. The method for soothing a cow udder of claim 1, further comprising the steps of:
supplying said menthol in an amount of 0.1-25.0 parts by weight of a total of said udder soothing mixture.

6. The method for soothing a cow udder of claim 1, further comprising the steps of:
supplying said tea tree oil in an amount of 0.1-25.0 parts by weight of a total of said udder soothing mixture.

7. The method for soothing a cow udder of claim 1, further comprising the steps of:
coloring to said udder soothing mixture.

8. A method for soothing a cow udder, comprising the steps of:
providing peppermint oil, menthol and tea tree oil;
providing a carrier;
providing an emulsifying agent;
combining said peppermint oil, said menthol, said tea tree oil, said carrier and said emulsifying agent with water to create an udder smoothing mixture;
spraying said udder smoothing mixture on at least one cow udder.

9. The method for soothing a cow udder of claim 8, further comprising the steps of:
mixing said peppermint oil, said menthol and said tea tree oil in said carrier to form a carrier mixture.

10. The method for soothing a cow udder of claim 9, further comprising the steps of:
mixing said carrier mixture in said emulsifying agent to form an emulsifying mixture.

11. The method for soothing a cow udder of claim 10, further comprising the steps of:
adding said water to said emulsifying mixture to form said udder soothing mixture.

12. The method for soothing a cow udder of claim 8, further comprising the steps of:
providing said carrier taken from the group of glycerin, humectants, glycols, alcohols, polyols and silicones, said carrier being 0.5-30.0 parts by weight of a total of said udder soothing mixture.

13. The method for soothing a cow udder of claim 8, further comprising the steps of:
supplying said emulsifying agent in an amount of 0.5-5.0 parts by weight of a total of said udder soothing mixture.

14. The method for soothing a cow udder of claim 8, further comprising the steps of:
supplying said peppermint oil in an amount of 0.1-25.0 parts by weight of a total of said udder soothing mixture.

15. The method for soothing a cow udder of claim 8, further comprising the steps of:
supplying said menthol in an amount of 0.1-25.0 parts by weight of a total of said udder soothing mixture.

16. The method for soothing a cow udder of claim 8, further comprising the steps of:
supplying said tea tree oil in an amount of 0.1-25.0 parts by weight of a total of said udder soothing mixture.

17. The method for soothing a cow udder of claim 8, further comprising the steps of:
providing Simulgel as said emulsifying agent.

18. A method for soothing a cow udder, comprising the steps of:
mixing peppermint oil, menthol and tea tree oil in a carrier to form a carrier mixture;
providing an emulsifying agent, said emulsifying agent being Simugel;
combining said carrier mixture and said emulsifying agent with water to create an udder smoothing mixture;
spraying said udder smoothing mixture on at least one cow udder.

19. The method for soothing a cow udder of claim 18, further comprising the steps of:
providing said carrier in an amount of 0.5-30.0 parts by weight of a total of said udder soothing mixture.

20. The method for soothing a cow udder of claim 18, further comprising the steps of:
providing said carrier taken from the group of glycerin, humectants, glycols, alcohols, polyols and silicones, said carrier being 0.5-30.0 parts by weight of a total of said udder soothing mixture.
